(19) United States
(12) Patent Application Publication
(10) Pub. No.: US 2019/0151047 A1
(43) Pub. Date: May 23, 2019

(54) ENDODONTIC RESTORATION POST

(71) Applicant: SYACT LLP, Plantation, FL (US)

(72) Inventors: George Bruder, West Palm Beach, FL (US); Sergio Kuttler, Plantation, FL (US); Anil Kishen, Mississauga (CA)

(21) Appl. No.: 16/091,654

(22) PCT Filed: Apr. 5, 2017
(86) PCT No.: PCT/US2017/026092

§ 371 Date: Oct. 5, 2018

Related U.S. Application Data
(60) Provisional application No. 62/318,548, filed on Apr. 5, 2016.

(51) Int. Cl. A61C 5/35; A61K 6/00

(52) U.S. Cl. CPC A61C 5/35; A61K 6/0044

(57) ABSTRACT

A structural member made at least in part from a fiber reinforced composite material is provided for use in strengthening the crown of an endodontically treated tooth. The structural member can be attached to a post member, such that the structural member and post member, in combination, define a 3D post for reinforcing an endodontically treated tooth.

### CROSS-REFERENCE TO RELATED APPLICATIONS

This application claims priority to U.S. App. No. 62/318,548, filed Apr. 5, 2016, the contents of which are incorporated herein by reference.

### STATEMENT REGARDING FEDERALLY SPONSORED RESEARCH OR DEVELOPMENT

Not Applicable.

### BACKGROUND OF THE INVENTION

This invention relates to dental posts, and in particular, to a 3-dimensional fiber post for use in restorations after endodontic treatment of a tooth.

Endodontic treatment begins with accessing the root canal(s) of a tooth by forming an access cavity AC in the tooth crown C to access to root canals R, as seen in FIG. 1. The treatment proceeds through cleaning, shaping, and then filling (obturation) of the canal(s) R. Once the treatment is finished, the access cavity must be closed to prevent recontamination. In some teeth, the remaining tooth above the gum line is lacking or compromised because of caries or trauma. In such treated teeth, a post is inserted into the root canal which extends above the level of the canal orifice (i.e., the opening into the root canal from the access cavity) to retain the core. A core is then secured to the post which helps replace missing or lacking tooth structure.

In many treated teeth, however, the tooth has an adequate number of walls which are sufficiently thick and sufficiently strong enough to support a filling. In such teeth, a post is not typically used, and the access cavity is filled with a filling material M, such as an amalgam or a resin, as seen in FIG. 2. Although, in some instances, a post P (FIG. 3) will be used to help stabilize the filling material. Posts made of a fiber composite (fiber posts) have been used.

Whether or not a post is used depends on remaining tooth structure after endodontic treatment has been completed (through obturation). If the coronal structures of a tooth are primarily intact, the primary preparation is the access cavity for endodontic treatment, and the tooth has favorable occlusion (e.g., an anterior tooth that has been traumatized but not fractured becomes non-vital), a routine restoration without a post would be indicated. When significant portions of the crown are missing, there may be the need to use a post to provide the coronal restoration with accessory retention. (See, Strassler, Fiber Posts: A Clinical Update, Mar. 7, 2007, Vol. 3, Issue 3, https://www.dentalaegis.com/id/2007/03/endodontics-fiber-posts-a-clinical-update.)

Fiber reinforced posts have an elastic modulus (or a material's stiffness) that more closely approximates that of dentin (which is about 18.6 GPa) allowing some slight flex in function, dissipation of stress and reduction of the likelihood of damage to the root. They thus have been shown to strengthen the root canal, and when stresses are placed on the tooth, the crown, rather than the root, may crack. While the fiber post reinforces the root structure, it does not reinforce the tooth's crown, and the tooth crown can still be prone to cracking and breaking. Those in the field have long been searching for a way to strengthen the filled tooth, but as of yet, no satisfactory method of strengthening restored/filled crown has been achieved.

### SUMMARY

Briefly stated, a structural member is provided for use in a cavity formed in a root canal or the crown of a tooth.

In accordance with one aspect, the structural member is a 3-D endodontic post comprising a post member (preferably made at least in part from a fiber material) having a lower portion shaped to be placed in a root canal and a top portion which, in use, extends into the access cavity formed in the tooth. An arm member (preferably made from a fiber material) extends from the top portion of the post member. The post member and arm member can be formed separately from each other, to be connected at the time of use, or can be formed as an integral, unitary one-piece product.

If the arm member is separate from the post member, it can be connected to the post member in several different manners. In one instance, the arm member comprises a bracket and the post member comprises an axially extending slot sized to receive the arm bracket. In another instance, the arm member comprises a head adapted to be snappingly connected to the post member. The head is generally circular or oval in shape, and defines either a closed shape which is sized to be received axially over the post or an open shape adapted to be received radially on the post. In this latter instance, the open shaped head can define an arc of about 180° to 360° to be snappingly received on the post. In another instance, post member has a hole formed in its top portion which is sized to snuggly receive the arm member. This opening can be a blind (closed) hole or a through hole. In one variation, the post top portion includes a second hole and the 3-D post includes a second arm member, wherein the second arm member defines an angle of other than 180° with the first arm member.

In accordance with an aspect of the arm member, the arm member includes texturing on a surface of the arm member. The texturing includes one or more of: axially extending and/or circumferential grooves, perforations through the arm, dimples (depressions) on the arm, projections on the arm surface, and roughening of the arm surface.

In accordance with another aspect, a structural member is provided for use in strengthening the crown of an endodontically treated tooth. The structural member is an elongate member having a length such that the structural member can be received horizontally in an access cavity of an endodontically treated tooth. The member is formed, at least in part, from a fiber composite.

In one aspect, the structural member includes a metal core about which the fiber composite material is formed. In one mode of manufacture, the fiber composite material and core are co-extruded to form the structural member.

In another aspect the structural member defines an arm member extending from a side surface of a post member. The arm member and post member can be formed independently of each other. In one variation, the arm member comprises a bracket and the post member comprises an axially extending slot sized to receive the arm bracket. In another variation, the arm member comprises a head adapted to be snappingly connected to the post member. The head is generally circular or oval in shape, and is defines either a closed shape which is sized to be received axially over the post or an open shape adapted to be received radially on the post. In this latter instance, the open shaped head defines an arc of about 180° to 360° to be snappingly received on the post. In another instance, post member has a hole formed in its top portion which is sized to snuggly receive the arm member. This opening can be a blind (closed) hole or a through hole. In one variation, the post top portion includes a second hole and the 3-D post includes a second arm member, wherein the second arm member defines an angle of other than 180° with the first arm member.

A method of strengthening the crown of an endodontically treated tooth comprises positioning a horizontal structural member made at least in part from a fiber composite horizontally in an access cavity of the tooth such that the structural member is spaced above, filling the access cavity with a filler material such that the structural member is surrounded by the filler material, and then curing the filler material.

In one aspect of the method, the horizontal structural member is part of a 3-dimensional post comprising a post member and the horizontal structural member extending from the post member. In this instance, the method comprises inserting the post member into a root of the tooth when the horizontal structural member is placed in the access cavity.

If the post member and structural member are independent pieces, the method includes a prior step of attaching the horizontal structural member to the post member, in accordance with any of the methods noted above.

As can be appreciated, the structural member (whether it is a single straight piece or a 3-D post) can include one or more of the above-noted aspects, in any desired combination.

### BRIEF DESCRIPTION OF THE DRAWINGS

FIG. 1 is a sagittal view of a tooth that has been shaped and cleaned and is prepared for obturation;

FIG. 2 is a sagittal view of a tooth that has undergone endodontic treatment and restoration;

FIG. 3 is a sagittal view similar to FIG. 2, but wherein a post was used in the restoration;

FIGS. 4A and 4B are elevational views of a fiber post that conforms to the morphology of a root canal following endodontic therapy for the post assembly;

FIG. 5A shows two arm members of a first style of arm member which is secured to the post;

FIG. 5B shows one of the arm members connected to the post;

FIG. 6 shows two variations of a second style of arm member that connects to the post;

FIG. 7 shows a post adapted to receive a third type of arm member;

FIGS. 7A and 7B show the post with one and two arm members, respectively, for use with Type III molars and Type III premolars, respectively;

FIG. 8 schematically shows the post assembly inserted in a tooth for restoration, with the boundaries of an access cavity shown in dotted lines;

FIG. 9 schematically shows the post assembly in two teeth, to demonstrate that the post can be inserted in the access cavity in different orientations.

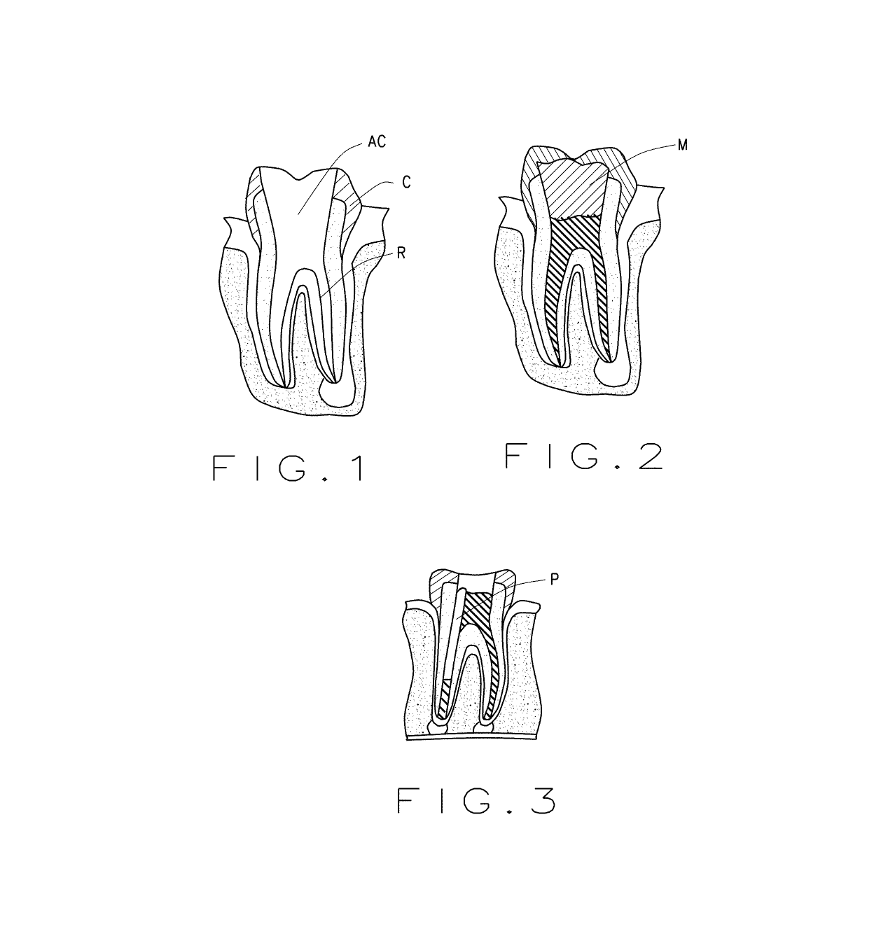

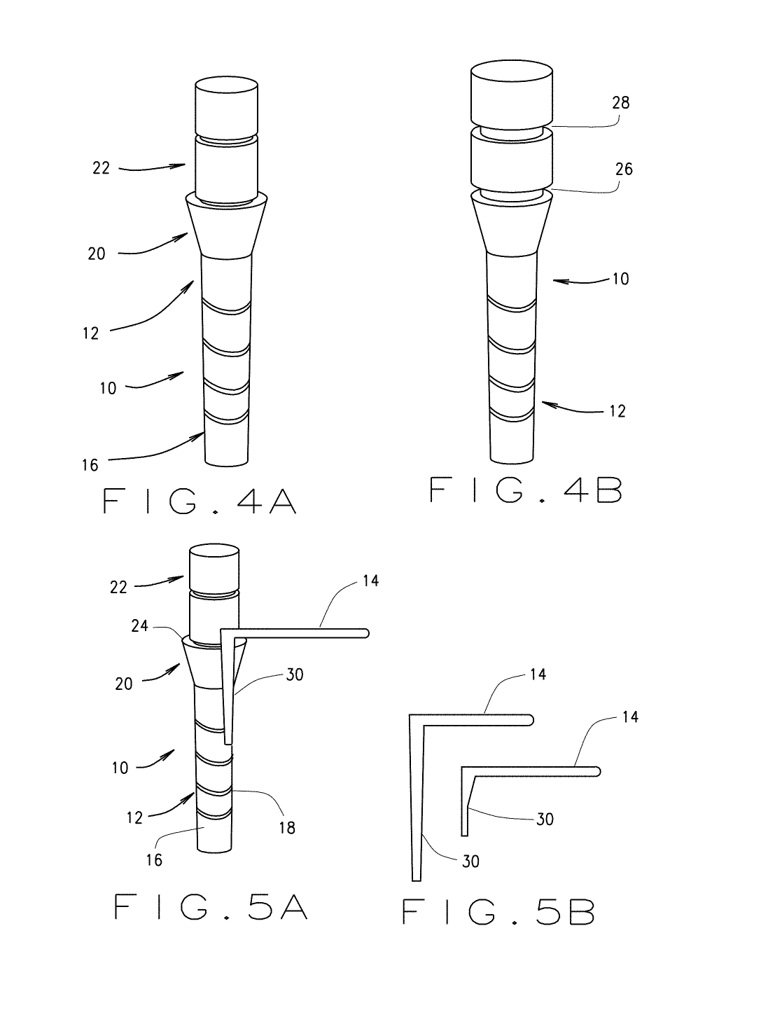

Corresponding reference numerals will be used throughout the several figures of the drawings.

### DETAILED DESCRIPTION

The following detailed description illustrates the claimed invention by way of example and not by way of limitation. This description will clearly enable one skilled in the art to make and use the claimed invention, and describes several embodiments, adaptations, variations, alternatives and uses of the claimed invention, including what we presently believe is the best mode of carrying out the claimed invention. Additionally, it is to be understood that the claimed invention is not limited in its application to the details of construction and the arrangements of components set forth in the following description or illustrated in the drawings. The claimed invention is capable of other embodiments and of being practiced or being carried out in various ways. Also, it is to be understood that the phraseology and terminology used herein is for the purpose of description and should not be regarded as limiting.

A 3-D post 10 comprises a post member 12 and an arm member 14 which extends from the post member to be substantially perpendicular to an axis of the post member 12. The post member 12 and arm member 14 are preferably both formed from fiber reinforced composites. The fiber reinforced composites can comprise, for example, carbon, glass silica, or quartz fibers in a resin, such as epoxy, bis-GMA, or other similar resins.

If the post member 12 and arm member 14 are formed as a unitary structure, the 3-D post can be milled from a block of fiber reinforced composite. If the post member and arm member are formed as separate pieces, they can be formed in several different ways. They can be milled from, for example, a rod made of the desired material. This rod can, for example, be extruded or molded, such as by injection molding. Further, if extruded or molded, the post member and arm member can be formed from a co-extrusion or co-molding. For example, the fiber composite can be co-extruded or co-molded about a metal core, which can be in the form of a wire or rod, and which can have a desired cross-sectional geometry. To add further strength to the post and/or the arm, the fibers can be braided or twisted about each other. Additionally, the post 10 can be 3D printed, which would allow for the post member and arm member to be formed as a one piece product. 3D printing would also allow for greater flexibility in the shape of the post and the length of the arm. The post could more closely correspond to the shape of the prepared canal and the arm could be sized to fit within the access cavity (as described below) without need for trimming. Similarly, molding of the post will allow for the post to be formed as a unitary, one-piece part wherein the post member and arm member are integrally formed. As can be appreciated, any other desired manufacturing method can be used to form the post, and the post can be formed as a unitary piece, in which the post member and arm member are integral, or the post can be formed form two pieces, which then requires that the post member and arm member be connected or joined together.

The post member 12 comprises bottom portion 16 which tapers slightly and has a helical groove 18 extending around the surface of the bottom portion. This bottom portion 16 can be round or generally oval in cross-section and is sized, as is known, to be received in a root canal. A mid-portion 20 extends upwardly from the top of the post's bottom portion 16. This mid-portion 20 is in the form of an upside down truncated cone (or a trapezoid). The side surface of the mid-portion 20 thus extends diagonally upwardly and away from the top of the post's bottom portion. An upper portion 22 extends upwardly from the top of the mid-portion 20. The post's upper portion is generally cylindrical, having a diameter less than the diameter of the top of the mid-portion 20. Thus, a floor 24 is formed at the top of the mid-portion and at the base of the top portion. The top portion includes lower and upper circumferential channels or grooves 26, 28, respectively. The lower channel 26 is at the level of the floor 24, and the upper channel 28 is formed about mid-way up the post top portion 22. As is known, when the post is inserted into a canal, the top of the mid-portion (i.e., the floor 24) will be approximately level with, or above, the floor of the access cavity AC in the crown. The post's top portion 22 extends into the access cavity when the post is installed in a tooth.

The arm member 14 extends outwardly (preferably radially outwardly) from the post top portion 22 slightly above or on the post floor 24 to increase resistance of the tooth crown to fracture. The length and diameter of the post arm 14 will vary depending on the tooth in which the post assembly is to be used. The arm 14 can have a length between about 3 mm and about 10 mm, but can be adjusted chairside by clinician, and a diameter of between about 0.2 mm and about 1.4 mm. The arm 14 is generally circular in cross-section, but can be define an oval (or an elongated circle) in cross-section. The arm 14 can even be generally rectangular or flat in cross-section. In addition, the surface of the arm 14 can be textured to increase retention and decrease dislodgement. Such texturing can include, for example, one or more of axially extending and/or circumferential grooves, perforations, dimples (depressions) and/or or projections that can be formed on the arm surface. The perforations could even extend through the arm (from one side of the arm to another). Alternatively, or in combination with grooves, perforations, dimples or projections, the surface of the arm can be roughened, such that the arm surface is not smooth.

The post member 12 and arm member 14 can be formed as a unitary member. However, the arm member 14 is currently formed independently of the post member and is connected to the post member 12 by the practitioner at the time of use of the 3-D post. Alternatively, as will be explained below, the arm 14 can be used in the pulp chamber with core or restorative material independently of the post. In FIGS. 5A and 5B, the arm member 14 is provided with a bracket 30 which slides into a slot of the post member 12 which extends axially downwardly from the post floor 24, such that the arm 14 is connected to the post member 12. The bracket 30 and post slot are shaped complementarily to each other, and are formed such that the arm 14 cannot be pulled radially from the post. For example, the bracket 30 can be circular, triangular, etc. In FIG. 5A, two arms 14 are shown with different length brackets 30.

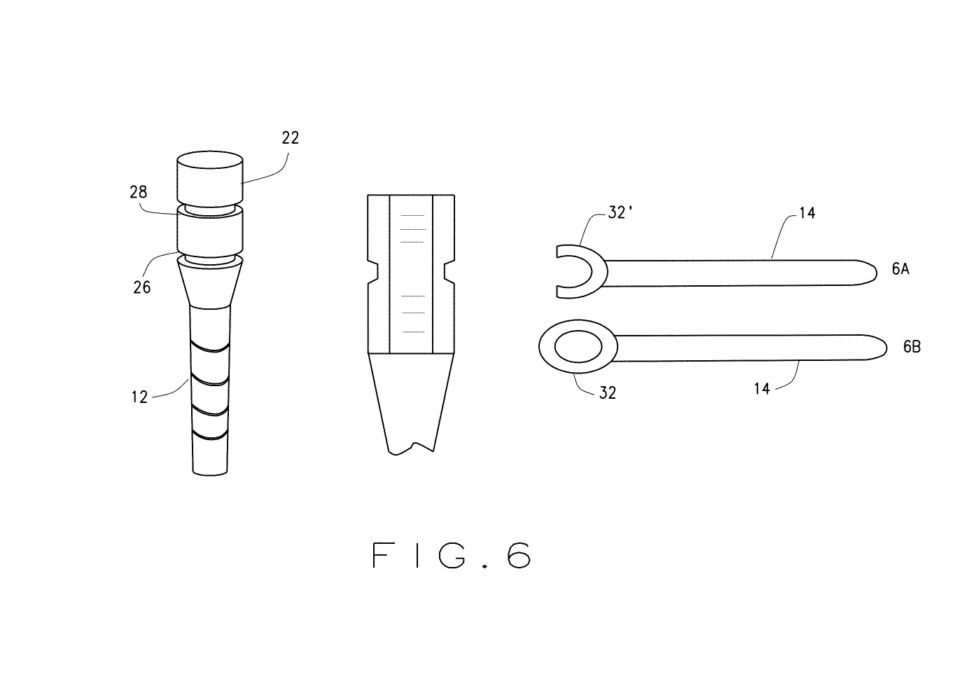

FIGS. 6A and 6B show alternative manners for connecting the arm member 14 to the post member 12. The arm 14 in FIG. 6A is provided with a generally oval-shaped head 32. The head 32 has in inner diameter and a width sized such that the head will snap into the lower groove 26 of the post top portion 22. In FIG. 6B, the arm 14 is provided with a head 32′ which defines a portion of a circle or oval at least slightly greater than 180°. The arm with the closed head 32 is applied axially to the post, whereas the arm with the open head 32′ is applied radially to the post.

The heads 32 and 32′ are both slightly elastic. The head 32 is sufficiently elastic such that the head can expand over the post top portion 22 to allow the arm to be applied axially to the post. The head 32 will then snap into the lower groove 26 when it engages the lower groove. The head 32′ is sufficiently elastic such that the ends of the “U” defined by the head will separate slightly to enable the head 32′ to be pushed radially onto the post top 22 at the level of the lower groove 26. As can be appreciated, the head 32 surrounds the post and the head 32′ partially surrounds the post. The heads 32 and 32′ are both made from the same fiber composite material as the arm 14. The heads 32 and 32′ thus structurally reinforce the post top portion 22.

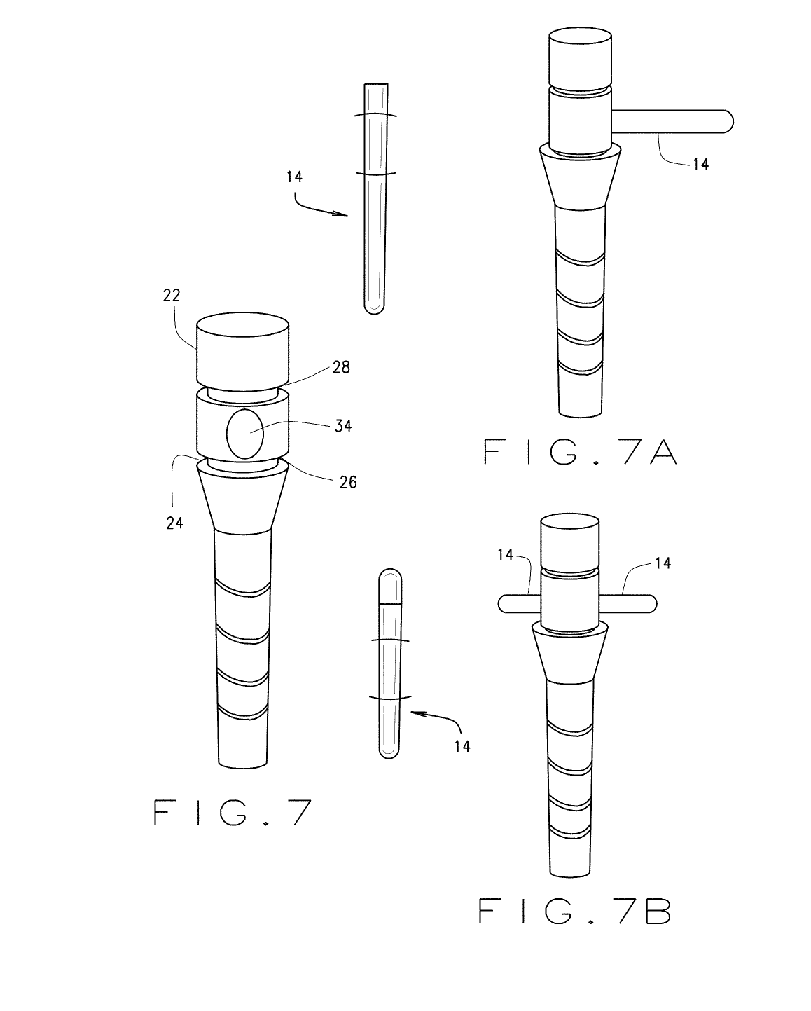

Lastly, FIG. 7 shows a hole 34 formed in the post top portion 22 between the channels 26 and 28. The hole 34 is sized and shaped to receive an end of the arm 14, and the arm 14 is press-fit into the hole 32. The hole 34 can be a blind hole (such that it does not extend through the post top portion) or can be a through hole. In FIG. 7A, the 3-D post 10 is shown with one arm 14, and in FIG. 7B, the 3-D post is shown with two arms 14. In FIG. 7B, the hole is a through hole, and the two arms are co-linear (and in fact are opposite sides of a one-piece arm). Alternatively, the post can be formed with two (or more) blind bores to receive two (or more) arms, in which case, define an angle of other than 180° (i.e., define either an acute or an obtuse angle).

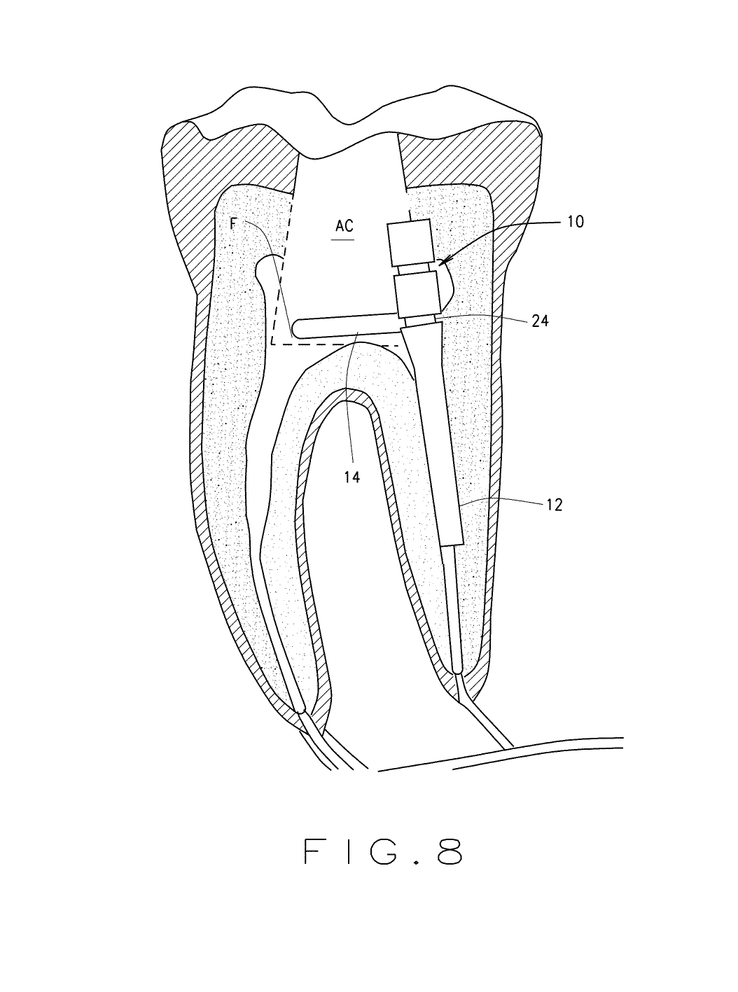

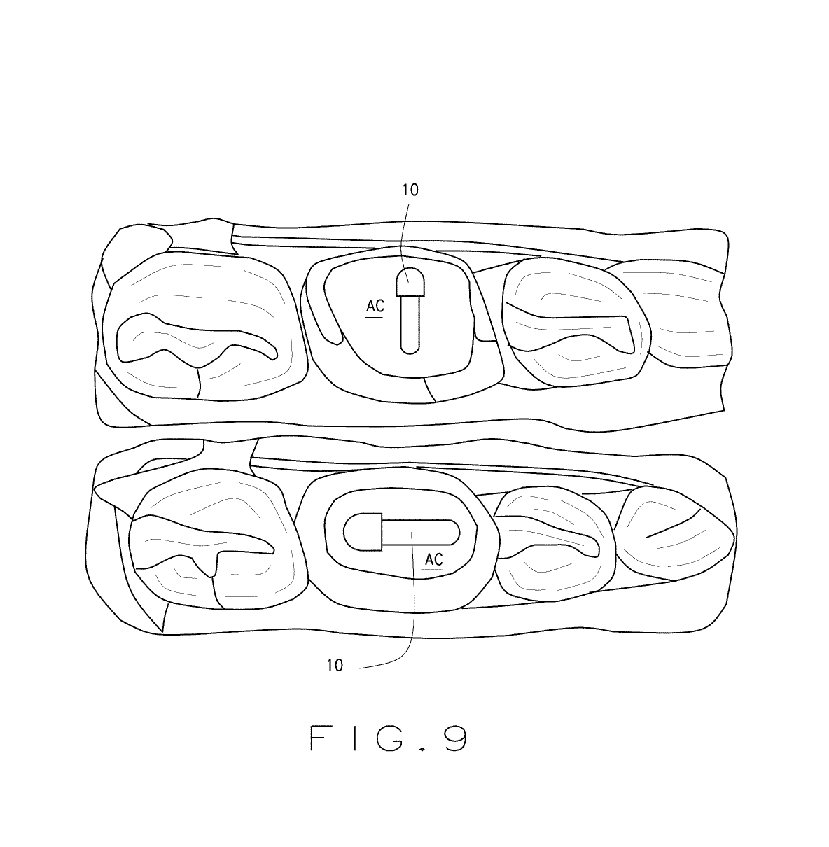

FIG. 8 schematically shows a 3-D post 10 inserted in a tooth. As seen, the post member 12 extends into the root canal R to a depth such that the post floor 24 is at or slightly above the floor F of the access cavity AC (shown in dotted lines).

To install the 3-D post 10 in an obturated root canal, a sufficient amount of the root canal filling is removed, for example, by heat or by drilling out, to enable the post member 12 to be inserted into the root canal R to the appropriate depth. Alternatively, the post can be installed after partial back packing during obturation. The 3-D post 10 is then inserted into the root to a depth such that the arm member 14 is above the access cavity floor. With the 3-D post in place (and after the root canal has been fully obturated), the access cavity AC is filled with a resin or other dental core material to form a core in the access cavity about the 3-D post (including the arm member 14). Once the resin has cured (such as by light and/or chemical), the remainder of the tooth crown can be built up to complete the restoration of the tooth.

As noted, the arm member 14 and the post member 12 are both made from a fiber reinforced composite. The fiber arm member 14 which is physically connected to the post member 12 increases the crown's flexural strength of the tooth crown in addition to the tooth root's flexural strength. The use of the arm 14 should therefore reduce the likelihood of crown breakage.

In a tooth wherein the root is too small to receive a post, or otherwise cannot receive a post, the arm 14 can be used independently of the post. In this instance, after obturation of an endodontically treated root canal, or after a pulp capping procedure, regenerative procedure or after other operative procedures are completed, an initial layer of core or other filling material can be laid down in the access cavity. The arm 14 is then set on top of this layer of core or filling material, and then the remainder of the access cavity is filled. Even though the post is not being used in this instance, the use of the arm 14 benefits the tooth in that it increases the flexural strength of the tooth (i.e., the tooth crown). This can be important during mastication, when large forces are applied to the tooth's crown.

Alternatively, some teeth can receive a post, but have a crown too small to receive the arm member 14. For such teeth, the post member 14 can be provided with the head 32 or 32′, but without the arm member 14. Using just the head would provide a washer-like (or part washer-like) structure around the upper portion 22 of the post member 12 and which has a diameter (or outer circumference) greater than the diameter (or outer circumference) of the post member upper portion 22. Hence, the head (without the arm member) would extend from the post member, and would thus still strengthen the tooth crown.

As various changes could be made in the above constructions without departing from the scope of the invention, it is intended that all matter contained in the above description or shown in the accompanying drawings shall be interpreted as illustrative and not in a limiting sense.

1. A 3-D endodontic post comprising
a post member having a lower portion shaped to be placed in a root canal and a top portion which, in use, extends into the access cavity formed in the tooth; and
an arm member extending from the top portion of the post member;
at least one of said post member and arm member being formed at least in part from a fiber reinforced composite material.

2. The 3-D endodontic post of claim 1 wherein the arm member is formed separately from the post member and is connected to the post member.

3. The 3-D endodontic post of claim 1 wherein the arm member comprises a bracket and the post member comprises an axially extending slot sized to receive the arm bracket.

4. The 3-D endodontic post of claim 2 wherein the arm member comprises a head adapted to be snappingly connected to the post member.

5. The 3-D endodontic post of claim 4 wherein the head is generally circular or oval in shape, and wherein the head is a closed shape and sized to be received axially over the post or an open shape adapted to be received radially on the post.

6. The 3-D endodontic post of claim 4 wherein the head defines an arc of about 180° to 360° to be snappingly received on the post.

7. The 3-D endodontic post of claim 1 including a hole formed in the top portion of the post member, said hole being sized to snuggly receive the arm member.

8. The 3-D endodontic post of claim 7 wherein the top portion includes a second hole and said 3-D post includes a second arm member, said second arm member defining an angle of other than 180° with the first arm member.

9. The 3-D endodontic post of claim 1 wherein said arm member includes texturing on a surface of said arm member.

10. The 3-D endodontic post of claim 9 wherein said texturing includes one or more of: axially extending and/or circumferential grooves, perforations through said arm, dimples (depressions) on said arm, projections on the arm surface, and roughening of the arm surface.

11. (canceled)

12. The 3-D endodontic post of claim 1 wherein the post member includes a metal core about which the fiber reinforced composite material is formed.

13. A structural member for use in strengthening the crown of an endodontically treated tooth: the member being an elongate member having a length such that the member can be received horizontally in an access cavity of an endodontically treated tooth; the member being formed, at least in part, from a fiber reinforced composite material.

14. The structural member of claim 13 wherein the member includes a metal core about which the fiber reinforced composite material is formed.

15. The structural member of claim 14 wherein said fiber reinforced composite material and core are co-extruded or co-molded to form said structural member.

16. The structural member of claim 13, wherein said structural member defines an arm member extending from a side surface of a post member.

17. The structural member of claim 16 wherein said arm member and post member are formed independently of each other.

18. The structural member of claim 17 wherein the arm member comprises a bracket and the post member comprises an axially extending slot sized to receive the arm bracket.

19. The structural member of claim 17 wherein the arm member comprises a head adapted to be snappingly connected to the post member.

20. The structural member of claim 19 wherein the head is generally circular or oval in shape, and wherein the head is a closed shape and sized to be received axially over the post or an open shape adapted to be received radially on the post.

21. The structural member of claim 19 wherein the head defines an arc of about 180° to 360° to be snappingly received on the post.

22. The structural member of claim 17 including a hole formed in the top portion of the post member, said hole being sized to snuggly receive the arm member.

23. The structural member of claim 22 wherein said hole is a through-hole, and said arm member extends through said through-hole to extend from opposite sides of said post member top portion.

24. The structural member of claim 22 wherein the top portion includes a second hole and said structural member includes a second arm member, said second arm member defining an angle of other than 180° with the first arm member.

25. The structural member of claim 13 wherein said structural member includes texturing on a surface thereof.

26. The structural member of claim 25 wherein said texturing includes one or more of: axially extending and/or circumferential grooves, perforations through said structural member, dimples (depressions) on said structural member, projections on the structural member surface, and roughening of the structural member surface.

27. A method of strengthening the crown of an endodontically treated tooth, the method comprising positioning a horizontal structural member made at least in part from a fiber reinforced composite material horizontally in an access cavity of the tooth; filling said access cavity with a filler material, such that said structural member is spaced above a floor of the access cavity and is surrounded by said filler material, and curing said filler material.

28. The method of claim 27 wherein said horizontal structural member is part of a 3-dimensional post, said post comprising a post member; said horizontal structural member extending from said post member; said method comprising inserting said post member into a root of the tooth when the horizontal structural member is placed in the access cavity.

29. The method of claim 28 comprising a step of attaching the horizontal structural member to the post member.

30. The method of claim 29 wherein said post member comprises an opening in a top portion thereof; said attaching step comprising inserting said horizontal structural member into said hole.

31. The method of claim 29 wherein said arm member comprises a head adapted to surround and/or snap onto said post member.

32. The method of claim 31 wherein said post member includes an axially extending slot and said arm member comprises a bracket portion sized to be received in said slot.
